(19) United States
(12) Patent Application Publication
(10) Pub. No.: US 2006/0211074 A1
(43) Pub. Date: Sep. 21, 2006

(54) Kinase-directed, activity-based probes

(76) Inventors: James P. Boyce, Kirkland, WA (US); Michael E. Brown, Kenmore, WA (US); Jeffrey N. Fitzner, Seattle, WA (US); Thomas J. Kowski, Seattle, WA (US)

Correspondence Address: OLYMPIC PATENT WORKS PLLC, P.O. BOX 4277, SEATTLE, WA, 98194-0277, US

(21) Appl. No.: 11/331,413

(22) Filed: Jan. 12, 2006

Related U.S. Application Data
(60) Provisional application No. 60/643,609, filed on Jan. 12, 2005.

(51) Int. Cl. C12Q 1/48; C07D 241/36; C07F 5/02

(52) U.S. Cl. 435/15; 544/353; 544/229

(57) ABSTRACT

Various embodiments of the present invention are directed to kinase-directed, activity-based probes (“KABPs”) that tightly bind to, and label, kinases. Each KABP includes a binding group that is recognized and bound by one or more kinases, a reactive group that tightly, and generally irreversibly, binds to the kinase, a tag group that labels the kinase, or that serves a chemical handle for subsequent procedures and processes, and a linker group that links the tag group to one or more of the reactive group and the binding group. Additional embodiments of the present invention are directed to methods for identifying kinases within, and isolating kinases from, living cells by use of one or more KABPs.

### CROSS-REFERENCE TO RELATED APPLICATION

This application claims the benefit of U.S. Provisional Application No. 60/643,609, filed Jan. 12, 2005.

### TECHNICAL FIELD

The present invention is related to synthetic chemical probes that target particular types of macromolecules and, in particular, to synthetic chemical probes directed to target macromolecules with kinase activity.

### BACKGROUND OF THE INVENTION

Kinases are enzymes that transfer phosphoryl groups from nucleoside triphosphate compounds, such as adenosine triphosphate, to acceptor molecules, including carbohydrates, proteins, nucleotides, and metabolic intermediates, such as oxaloacetate. Protein kinases, which transfer phosphoryl groups from nucleoside triphosphates to threonine, serine, and tyrosine residues of catalytic and regulatory proteins, are important components of many different cell-cycle-regulating systems as well as intracellular and intercellular communications systems involved in development, normal cell function, gene-expression regulation, and the onset and development of pathological conditions, including cancer. Over 500 different kinases have been discovered. Protein kinases may be directly or indirectly activated by various stimuli, including hormones, neurotransmitters, and growth factors, and may, in turn, activate myriad different types of proteins and other biopolymers, often in a series of cascading reactions that vastly amplify the original stimuli.

Because of their importance in contributing to a variety of pathologies, including cancer, inflammatory conditions, autoimmune disorders, cardiac diseases, neoplasia, cell proliferation and invasion, tumor-associated angiogenesis, and metastasis, protein kinases are attractive targets for research and drug development. Pharmaceutical companies continue to seek small-molecule-drug inhibitors of, and therapeutic agents directed to, particular protein kinases for study and treatment of various types of diseases. In addition, pharmaceutical companies are eager to identify new kinases, and new signaling pathways or other cellular activities mediated by the new kinases, as new targets for therapeutic drugs. Researchers and drug developers also seek ways to evaluate candidate therapeutic drugs to identify unintended interactions with kinases to which the candidate therapeutic drugs are not targeted. Unintended interactions between a candidate therapeutic drug and non-targeted kinases may lead to serious side effects that limit the usefulness of the candidate therapeutic drug, or, at least may lead to research into investigating therapeutic regimes, drug-delivery techniques, or chemical modifications of the candidate therapeutic drug to ameliorate the side effects. Evaluation of potential unintended interactions between candidate therapeutic drugs and kinases is particularly important in view of the large number of different types of kinases, the large amplifications of kinase-based signals, the wide ranging and profound effects of kinase activity on cellular organization and processes, and the large number of kinase molecules active within cells at any given time.

FIG. 1 is a cut-away view of the contents of an animal cell. The cell 102 is enveloped in a phospholipid-bilayer plasma membrane 104 that prevents free exchange of water and water-soluble small-molecule organic compounds, inorganic salts, ions, and macromolecules, between the external environment of the cell and the interior of the cell. A large variety of transport and pore proteins are embedded in the plasma membrane to facilitate specific exchange of molecules between the external environment of the cell and the interior of the cell, often accompanied by expenditure of chemical energy by the cell to transport the molecules against unfavorable chemical gradients, and many receptors and signaling proteins are associated with the cell membrane to transform external chemical signals and stimuli into internal, cellular signaling systems. The cell includes a nucleus 106 surrounded by a membranous nuclear envelope 108, mitochondria, such as mitochondrion 110, also surrounded by membranes, additional membrane-enveloped organelles, and other membranous structures, such as the endoplasmic reticulum 112 and the Golgi apparatus 114.

Kinases, and other therapeutic drug targets, may be located in the cytosol 116, a fluid environment within cells, may be located within intracellular, membrane-enclosed organelles, such as the nucleus 106 and mitochondria 110, or may be associated with membranes or membranous structures. Often, therapeutic drugs that either passively diffuse into cells, or that are actively transported into cells by transport proteins associated with cell membranes, may not end up being uniformly distributed throughout a cell, but may, for example, be concentrated in membranous structures, in the cytosol, or closely associated with biopolymers that have specific locations within the cell. Thus, it cannot be assumed that a particular kinase is exposed to a particular drug within a cell, despite general active transport or passive diffusion of the drug into the cell.

FIG. 2 shows the van der Waals surface of a portion of a kinase. Kinases, like other enzymes and globular proteins, comprise of one or more polypeptide polymers that generally spontaneously fold and self-associate, during and after synthesis, or that fold under the influence of chaperones or due to other programmed influences, to produce one or a few stable conformations in a particular chemical environment. In general, the chemical environment for proteins and other enzymes is an aqueous, concentrated, and complex solution, as found in the cytosol or in various organelle matrices, or a more hydrophobic environment in which the enzyme or globular protein is closely associated with membrane lipids or with other proteins. The catalytic power of kinases, as with most enzymes, depends on the three-dimensional conformation of the protein. Normally, an enzyme has one or more binding sites at which one or more substrates of the reaction catalyzed by the enzyme specifically bind. For example, in FIG. 2, a cleft, or pocket 202, in the kinase 200 includes two binding sites for the two substrates for the phosphoryl-transfer reaction catalyzed by the kinase. Each different kinase recognizes and binds to at least two specific substrates. In general, the binding specificity is sufficiently high that only a few, very closely related naturally occurring compounds are recognized by, and bound by, a particular kinase at each of the two binding sites. Substrate binding is mediated by the overall shape and size of the cleft or pocket containing the binding domains, as well as by numerous non-covalent interactions between a substrate and amino-acid side chains, polypepyide-backbone, amide nitrogen atoms and carbonyl oxygen atoms, and terminal carboxyl and amino groups that line the pocket or cleft or that protrude into the pocket or cleft. These interactions include ionic, electrostatic, and van der Waals interactions, hydrogen bonding, and entropy increases associated with minimizing exposure of hydrophobic portions of a substrate and hydrophobic amino-acid side chains of the kinase to water molecules. In some cases, the conformation of the kinase may be altered upon binding of one or both substrate molecules, facilitating stable association of the substrate and kinase, and facilitating catalysis of the phosphoryl-transfer reaction catalyzed by the kinase. In addition to substrate-binding domains, kinases are often allosteric proteins, and include regulatory binding domains at which various small-molecule regulators or portions of biopolymer regulators may bind to, and alter the conformation of, the kinase, in turn altering the catalytic activity of the kinase. As with substrate-binding domains, allosteric regulator-binding domains have high specificities for particular, closely related small-molecules and portions of biopolymers. Kinases catalyze reactions by increasing reaction rates due to localized concentration effects, selecting and restricting orientations of substrates, by stabilizing transition states of reactions and lowering the free-energy barrier for the reaction, and by participation of amino-acid side chains as proton donors, electron acceptors, and nucleophilic intermediates in the reaction.

Many of the techniques commonly employed to identify and isolate kinases from biological tissues for drug discovery and candidate-drug-evaluation research involve homogenizing tissues, lysing cells, and employing various separation and isolation techniques to identify and isolate kinases from cell-extract solutions. FIG. 3 illustrates commonly used approaches to isolating and identifying particular kinases. First, a tissue is mechanically or mechanically and chemically homogenized to produce a crude cell extract solution 302. The solution is then centrifuged in a high-speed centrifuge to separate soluble proteins from membrane fragments, chromatin fragments, and other materials produced by disrupting intact cells. Different types of soluble proteins are separated from one another, by different types of chromatography techniques 306, by gel electrophoresis techniques 308, or by microarray-based techniques 310. In chromatography techniques, a complex solution of soluble proteins is passed through a column 312 containing a chemical matrix, which interacts differently with different types of proteins, leading to elution of different types of proteins from the column at different points of time, or in different fractions of a total volume of solution eluted from the column. In gel electrophoresis, proteins migrates, under an applied electric field, through a slab of gel, with mobilities generally depending on molecular weight and other factors, leading to separation of different types of soluble proteins into bands, such as band 314. A microarray is a dense, two-dimensional matrix, each cell of which contains a different probe molecule bound, to the surface of the microarray, that specifically binds to, or recognizes, one or a few closely related target molecules. When a microarray is exposed to a complex solution of different types of soluble proteins, probe molecules within a particular cell may each bind a particular type, or closely related types, of soluble proteins within each cell of the microarray. Various techniques can be used to instrumentally detect soluble proteins in elution fractions, gels, or bound to the surface of microarrays, including spectrophotmetry, detection of fluorescent, phosphorescent, or radioactive signals emitted by chemically or radioactively labeled proteins, by mass spectroscopy, and by other techniques.

In some cases, the different, isolated proteins may be recognized as kinases by assaying their ability to catalyze phosphoryl transfer reactions. In other techniques, such as affinity chromatography, or microarray-based techniques, the location of a soluble protein within an elution fraction or at a particular point on a microarray may be indicative of the protein's ability to bind kinase substrates. Once kinases are identified, similar techniques, carried out on larger volumes of cell extract, may be used to isolate and purify sufficient quantities of kinases in order to assay the kinase for binding of particular candidate therapeutic drugs.

Although the commonly employed techniques, discussed above with reference to FIG. 3, have been used for many years to identifying kinases, for assaying kinases for specific interactions with candidate drugs, and for detecting unintended interactions of non-targeted kinases with drugs or other molecules, these techniques have significant shortcomings. First, when cells are disrupted in order to prepare cell extracts, many kinases that, in an intact cell, inhabit a particular, local environment within the cell end up in solution with many other molecules and cellular components with which the kinases may not normally interact in the intact cell. For example, a kinase may be subject to degradation by various kinases-degrading enzymes, and be degraded during the biochemical separation and identification processes by kinase-degrading enzymes that the kinase would be exposed to only at the end of its life, and not during its normal function. As another example, the kinase may be exposed to regulatory molecules that the kinase would not normally be exposed to in its local environment within the cell, and may be inhibited or activated by these regulatory molecules, and thus not show the phosphorylation activity in the cell extract, and in subsequent, purified solutions, that the kinase normally exhibits in the normal cellular environments. Kinases may be inadvertently denatured, and irreversibly lose their native three-dimensional conformations, at various interfaces and boundaries encountered during the separation and isolation procedures, including at the surface of a microarray and at solution/air, solution/glass, and solution/matrix interfaces. Countless other types of interactions and environmental conditions not encountered by the kinase in its normal state within a cell may lead, during separation and isolation procedures, to loss of kinase activity or loss of kinases all together.

Drug developers, researchers, and other scientific and technical personnel that need to identify kinases and evaluate kinase activity within living organisms therefore have recognized a need for better techniques to identify and isolate kinases, and other types of catalytic biopolymers, in order to identify new targets for drug therapy, as well as to evaluate candidate drugs for unintended interactions with kinases to which they are not directed.

### SUMMARY OF THE INVENTION

Various embodiments of the present invention are related to kinase-directed, activity-based probes (“KABPs”) that bind to, and label, kinases. Each KABP includes a binding group that is recognized and bound by one or more kinases, a reactive group that tightly, and generally irreversibly, binds to the kinase, a tag group that provides a detectable label for the kinase-KABP pair, or that serves as a chemical handle for subsequent procedures and processes, and a linker group that links the tag group to one or more of the reactive group and the binding group, spacing the tag group from the reactive and binding groups. Additional embodiments of the present invention are directed to methods for identifying kinases within, and isolating kinases from, living cells by use of one or more KABPs.

### BRIEF DESCRIPTION OF THE DRAWINGS

FIG. 1 is a cut-away view of the contents of a cell.

FIG. 2 shows the van der Waals surface of a portion of a kinase protein.

FIG. 3 illustrates commonly used approaches to isolating and identifying particular kinases.

FIG. 4 illustrates one method, representing an embodiment of the present invention, that uses a KABP or several different types of KABPs to label kinases within an intact cell.

FIGS. 5A-F abstractly illustrate recognition and binding of a KABP by a target kinase.

FIG. 6 shows a generalized, schematic representation of kinase-directed, activity-based probes that represent embodiments of the present invention.

FIGS. 7A-F show the chemical structures of seven kinase-directed, activity-based probes that represent exemplary embodiments of the present invention.

FIGS. 8A-C show three generalized chemical formulas for three classes of kinase-directed, activity-based probes, two of which include the specific probe embodiments shown in FIGS. 7A-F, that represent embodiments of the present invention.

FIG. 9 shows a number of different small-molecule inhibitors, including known kinase inhibitors, that may be used, either in the forms shown in FIG. 9, or in derivative forms, as binding groups for alternative KABP embodiments of the present invention.

FIG. 10A illustrates a general approach for synthesis of a variety of different anilinoquinazoline moieties that can serve as binding groups within kinase-directed, activity-based probes that represent embodiments of the present invention.

FIGS. 10B-10N show a number of different N⁴-substituted quinazoline-4,6-diamines produced by the synthetic steps shown in FIG. 10A.

FIGS. 11A-B illustrate several alternative synthetic methods for synthesizing reactive-groups/linker-group moieties included in kinase-directed, activity-based probes that represent embodiments of the present invention.

FIG. 12 shows final synthetic steps used to assemble an exemplary kinase-directed, activity-based probe that represents one embodiment of the present invention.

### DETAILED DESCRIPTION OF THE INVENTION

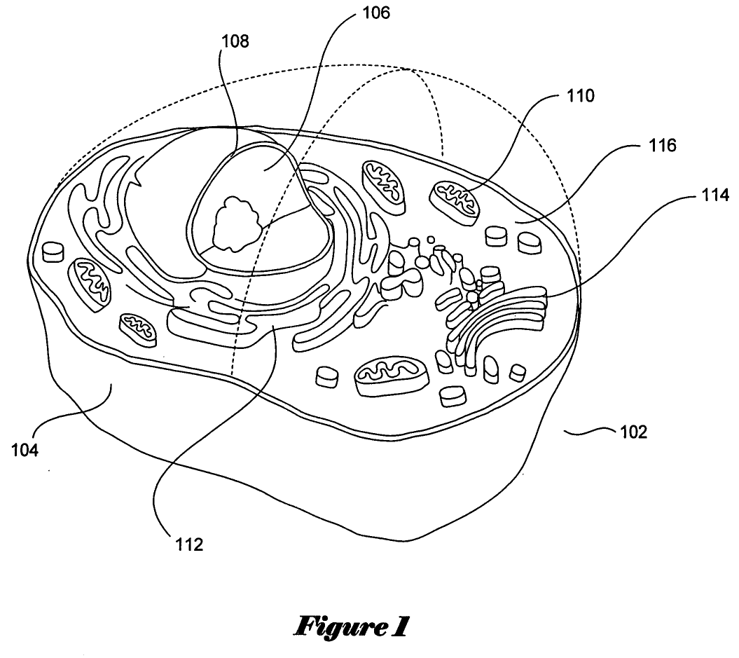

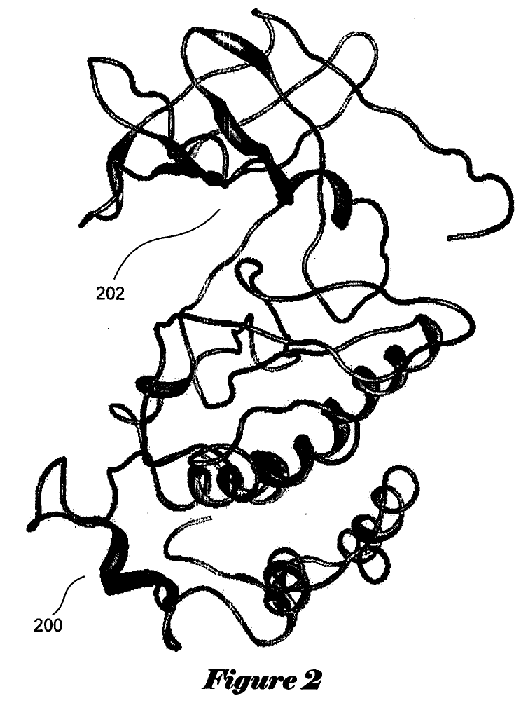

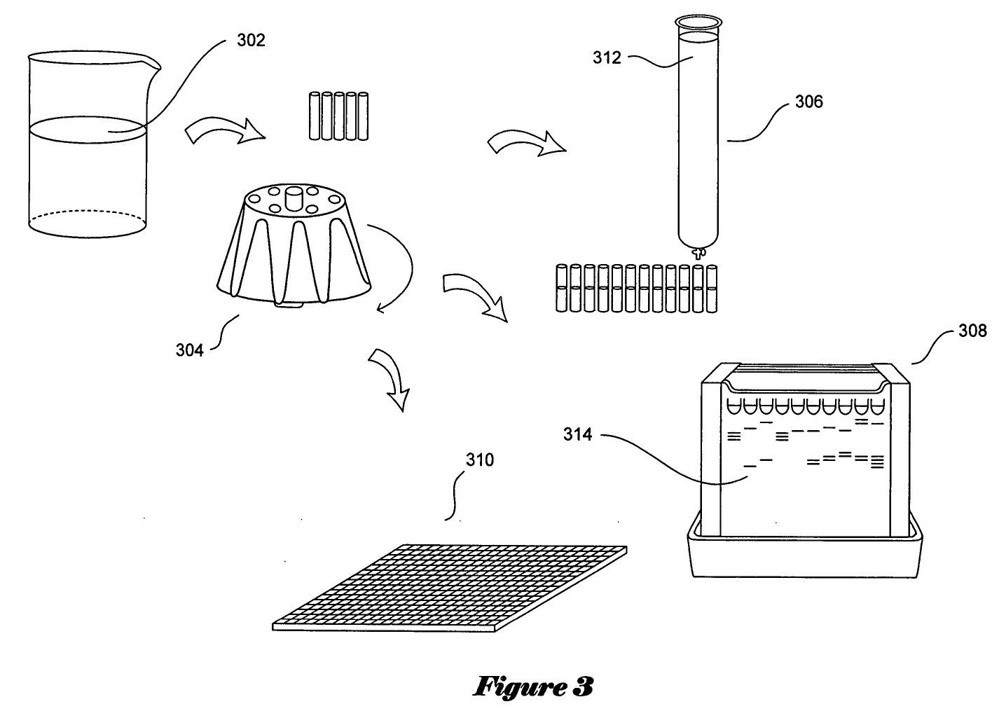

Embodiments of the present invention are directed to kinase-directed, activity-based probes (“KABPs”) that can be used to label kinases within living cells for a number of different purposes, including subsequent identification, separation and purification, and characterization of kinases. As discussed in the previous subsection, there are many traditional biochemical techniques that can be used to identify kinases present in cell extracts, to separate and purify particular types of kinases from cell extracts, and to characterize kinases isolated from cell extracts. However, disruption of cells may result in degradation, denaturization, and inhibition or activation of kinases. Moreover, kinases present at only very low concentrations within cells may be difficult or impossible to identify in complex cell-extract solutions by these techniques. As discussed above, with reference to FIG. 1, various types of kinases may be found only in local, specialized environments within cells, with activities dependent on maintenance of the local environments, and with accessibilities to small molecules and biopolymers strongly influenced by the local environments. These local environments are not preserved when tissues are homogenized and cells disrupted to produce cell-extract solutions from which soluble proteins are generally harvested and identified.

The above-mentioned problems may acutely impact drug-discovery and candidate-drug-evaluation research. Kinases are often involved in signal-amplification cascades within a cell, in which a receptor, receptor-associated, or receptor-stimulated kinase phophorylates a second-tier protein kinase, initiating a complex cascade of activation of increasing numbers of kinases that eventually activate enzymes or phosphorylate a small-molecule messenger, leading to significant metabolic, transcriptional, and/or cell-cycle-related responses by the cell. The initial kinases in the cascade may be present in only a very few copies per cell, and thus may be difficult to identify and isolate from complex cell-extract mixtures. The problem of disruption of local environments of kinases is particularly significant when evaluating non-target interactions between candidate therapeutic drugs and kinases. It may be the case that, in an intact cell, a candidate therapeutic drug would not reach a concentration within a local environment of a kinase sufficient to inhibit, activate, or be modified by the kinase under normal therapeutic regimes. However, removed from the local environment that the kinase normally occupies within the cell, and exposed to the candidate therapeutic drug, the kinase may show a significant interaction with the drug, leading to a false positive conclusion. Conversely, a kinase may be deactivated, degraded, or denatured during separation and purification procedures, and would otherwise have interacted with the candidate drug, leading to a false negative conclusion.

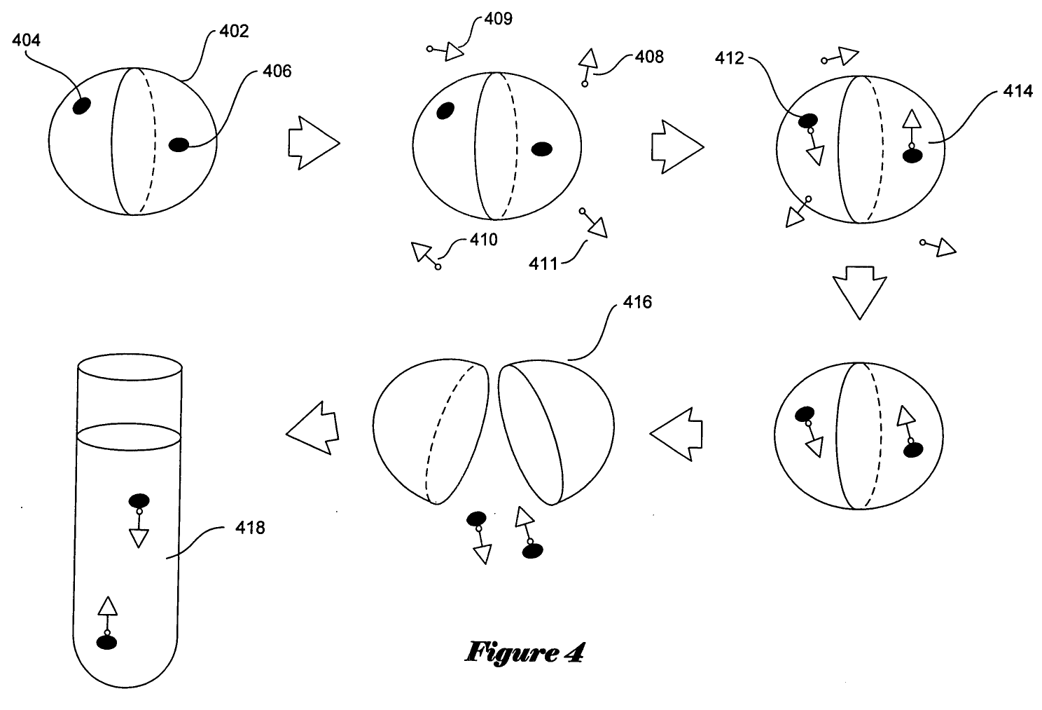

FIG. 4 illustrates one method, representing an embodiment of the present invention, that uses a KABP or several different types of KABPs to label kinases within an intact cell. As shown in FIG. 4, in schematic fashion, an intact cell 402 may contain several kinases of interest 404 and 406 that occur within particular, local environments within the cell. The intact cell may be exposed to a solution of one or more types of KABPs 408-411. The KABPs, to which the intact cell is exposed, pass through the cell membrane and are tightly bound to the kinase targets to form kinase-KABP pairs 412 and 414. The intact cell can then be rinsed, to remove remaining, extracellular KABPs, and the cell may then be lysed 416 in order to extract the kinase-KABP pairs into a solution 418 that can be processed by various biochemical techniques, and subject to analytical methods that reveal the presence of the kinase-KABP pairs, or to isolate and purify particular kinase-KABP pairs. For example, a KABP that contains a fluorescent tag group may produce an easily detectible, optical signal upon illumination of a sample containing the KABP by light of a frequency equal to the energy needed to excite the fluorescent tag group, allowing for instrumental detection of even tiny concentrations of the kinase-KABP pairs. Alternatively, the tag group may contain a radioisotope, allowing for detection of KABPs in solution by detection of emitted radiation. In yet additional alternative KABPs, the tag may be chemiluminescent, or an intermediate in a chemiluminescence-producing reaction, or may contain elements of particular atomic masses that can be readily detected by mass spectroscopy. Alternatively, the tag group may be a kind of chemical handle that can be recognized and bound by compounds or materials in subsequent separation processes. For example, the tag group may have a strong affinity for an affinity-chromatography matrix, allowing the kinase tightly bound to the KABP to be isolated and purified using affinity chromatography techniques. Of course, in actual kinase-labeling procedures, many hundreds of thousands to millions of cells may be exposed to KABP solutions, each cell containing extremely large number of potential target kinases.

Because KABPs bind to the kinase within an intact cell, and generally bind irreversibly, through a covalent bond, kinase-KABP conjugates can be subsequently detected, following disruption of the cell, despite a variety of events that would otherwise deactivate the kinase. Generally, only an active kinase binds a KABP, since the KABP binding group mimics a kinase substrate. In cases where KABP is not encountered by the kinase in the local environment which the kinase occupies within the cell, and provided that unbound KABP can be removed from the cell, or scavanged during homogenization and lysing by a chemical compound introduced for that purpose, the absence of interaction between a kinase and a KABP introduced into the intact cell may be indicative of the lack of activity within an intact cell under the experimental conditions.

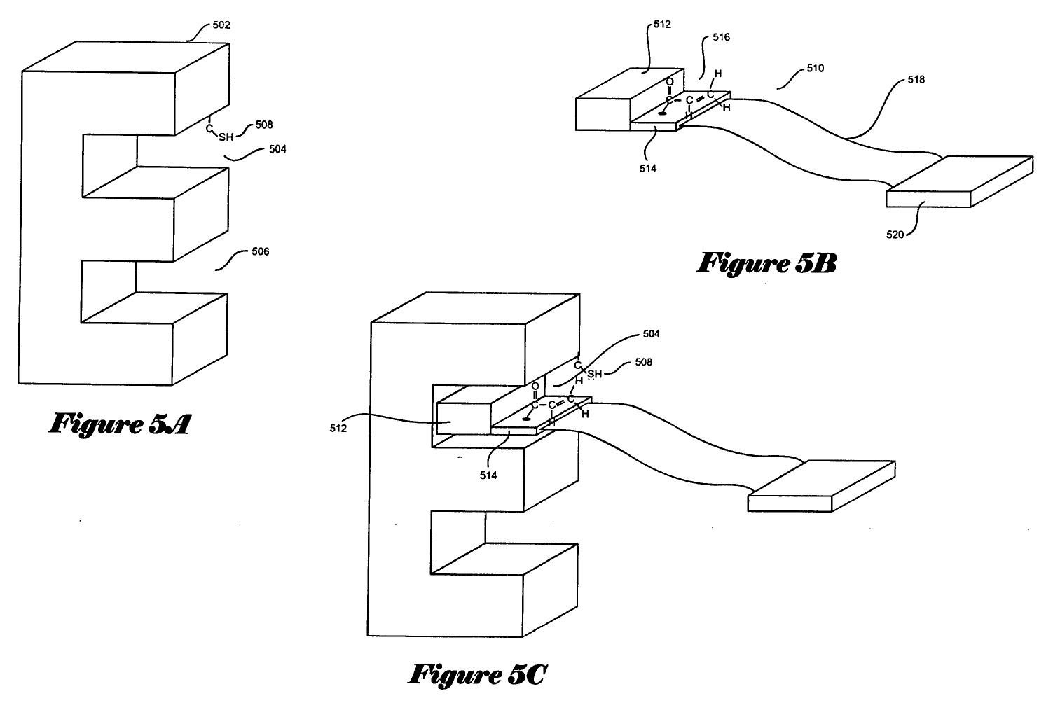

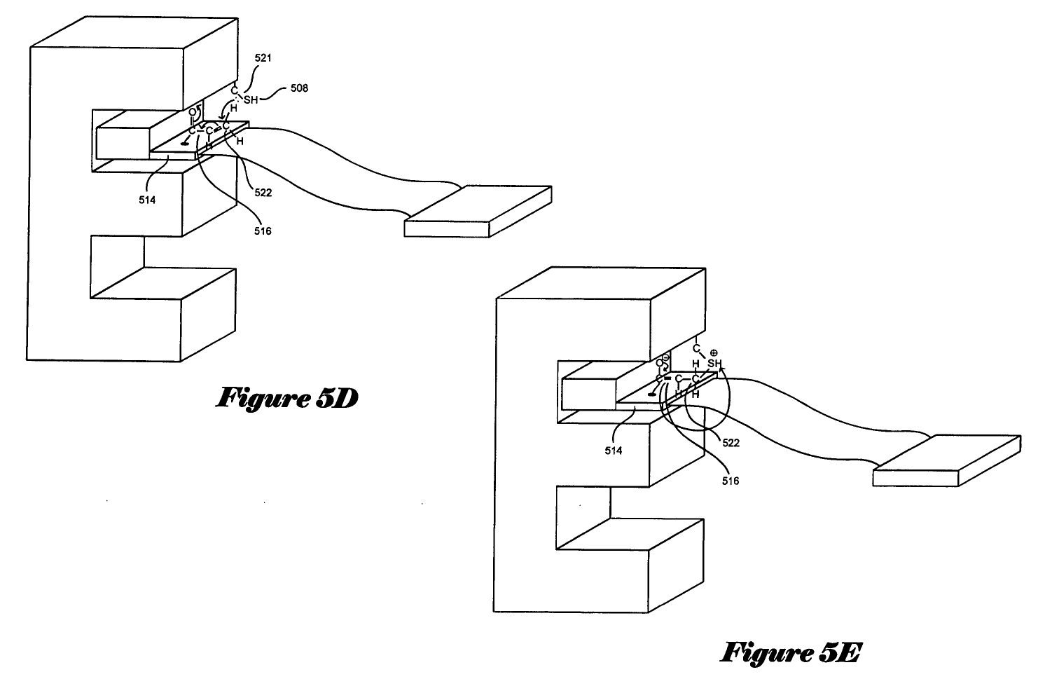

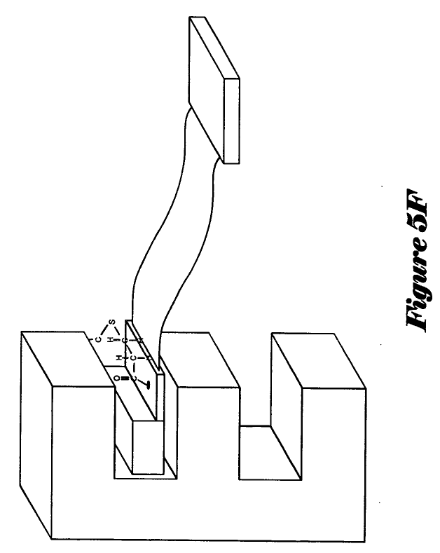

FIGS. 5A-F abstractly illustrate recognition and binding of a KABP by a target kinase. FIG. 5A shows a schematic representation 502 of the kinase. The kinase may include multiple binding domains 504 and 506 represented in FIG. 5A as slots or invaginations within the kinase. Various amino-acid side chains and backbone carbonyls and amide nitrogens line the surfaces of the binding domains, and provide a highly defined, three-dimensional electrochemical surface with high affinity for one or a family of closely related chemical compounds such as, in the case of kinases, nucleoside triphosphates, small-molecule substrates phosphorylated in the phosphoryl-group transfer reaction catalyzed by the kinase, or specific portions of macromolecules phosphorylated by the kinase. Other binding sites, such as binding site 506, may have strong affinities for various small-molecule regulators or portions of biopolymer regulators that, upon binding, may induce conformational changes throughout the kinase, affecting the binding affinity of the kinase for substrates and/or affecting the catalytic activity of the kinase. In the schematic representation of the kinase shown in FIG. 5A, a cysteinyl sulfhydryl group 508 is shown extending into the binding domain 504. This cysteinyl sulfhydryl group may or may not be involved in normal substrate binding or in the phosphoryl transfer reaction catalyzed by the kinase.

FIG. 5B shows a schematic representation of a KABP that targets the kinase schematically shown in FIG. 5A. The KABP 510 includes a binding group, or binding moiety, 512 that is bound by the kinase in the binding domain (504 in FIG. 5A). Note that a KABP may target either a substrate binding domain or an allosteric regulator binding domain. The KABP 510 includes a reactive group, or reactive moiety, 514. The reactive group, shown in FIG. 5B, includes a chemically reactive moiety 516, in the case of the KABP shown schematically in FIG. 5B, an acrylyl moiety. The KABP also includes a linker group, or linker moiety, 518 that is relatively chemically unreactive and with appropriate conformational flexibility to provide reasonable permeability in cell membranes, but with sufficient rigidity to maintain separation between the reactive and binding groups and a tag group, or tag moiety, 520 that acts as an instrumentally detectable label for subsequent identification or as a chemical handle during subsequent purification processes. The linker group 518 serves to prevent the tag group 520 from interfering with binding of the binding group 512 to the binding domain within the kinase, and may also serve to allow the tag group to remain at a position exterior to, or on the surface of, the kinase following binding of the KABP to the kinase, so that the tag is accessible as a chemical handle in subsequent purification steps, or so that the tag is not specifically associated with a kinase moiety that can quench emission from excited states of the tag, or otherwise compromised as a label.

As shown in FIG. 5C, when the KABP is introduced into the environment of the kinase, and the kinase is active, the kinase binds the binding group 512 of the KABP within the binding domain 504. Binding of the binding group by the kinase positions the reactive group 514 in close proximity to the reactive cysteinyl sulfhydryl group 508, in the example of FIGS. 5A-F. The sulfur atom 521 of the cysteinyl sulfhydryl group 508 then acts as a nucleophile and attacks the distal carbon 522 participating in the unsaturated bond of the acrylyl group 516, resulting in formation of a covalent bond, as shown in FIG. 5E-F, by a Michael addition. Once the covalent bond is formed, the KABP is irreversibly bound to the kinase, forming a stable kinase-KABP pair that can survive many different types of subsequent isolation, purification, and other chemical and biochemical processes.

The acrylyl moiety used as an exemplary reactive group in the example of FIGS. 5A-F is but one example of the many different possible types of reactive groups that may be employed to essentially irreversibly bind a KABP to a kinase. There are many possible KABP-reactive-group/kinase-functional-group interactions that can lead to the desired, effectively irreversible binding needed for stable KABP labeling of kinases, with suitabilities, in part, dependent on the specific kinase. Although covalent bonds are one example of a means to achieve an essentially irreversible bonding of a KABP to a kinase, non-covalent interactions between the reactive group and kinase functional groups may cooperatively produce a sufficiently large association constant for a kinase-KABP complex to allow for robust labeling of the kinase by the KABP in certain applications. In general, any type of KABP-kinase association may be reversible under selected chemical conditions. The term “irreversible” indicates that the association is sufficiently stable with respect to the processes and procedures subsequently employed to study the KABP-kinase conjugate. Similarly, many different small-molecule substrate analogs can generally be identified for incorporation into a KABP designed to target a particular kinase or class of kinases, and a wide variety of different tag groups and linker groups can be used.

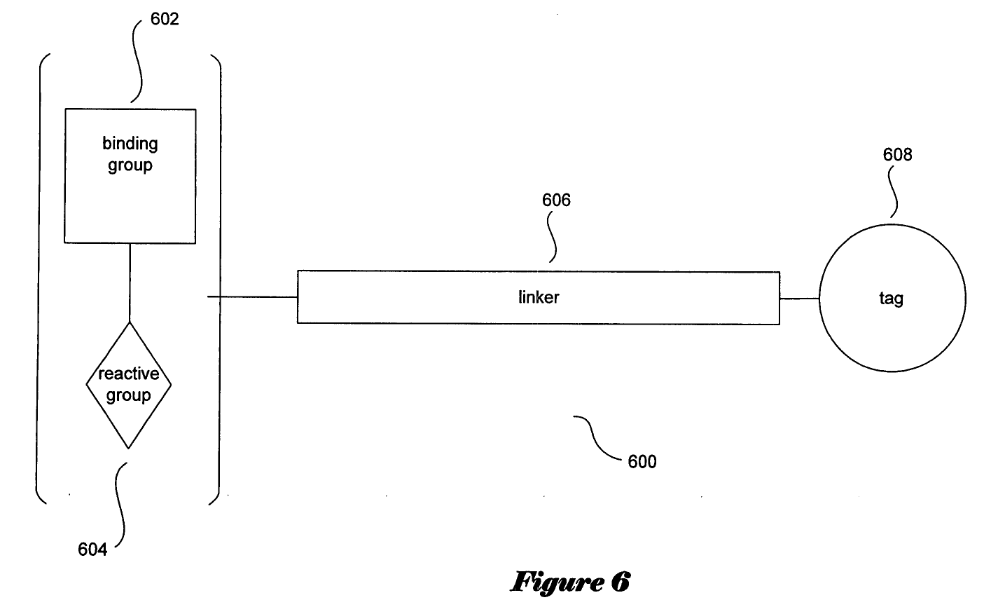

FIG. 6 shows a generalized, schematic representation of kinase-directed, activity-based probes that represent embodiments of the present invention. As discussed previously, a KABP 600 includes: (1) a binding group 602 that is bound by one or more target kinases; (2) a reactive group 604, that tightly binds, generally covalently, a target kinase in order to irreversibly bind the KABP to the kinase; (3) a linker group 606 that serves as an internal spacer; and (4) a tag group 608 that serves as a chemical handle or instrumentally detectable label for the kinase-KABP pair.

Binding groups may have different characteristics specifically selected for different applications and uses of KABPs. In the case that a KABP is used in a method to identify new kinases, or to identify kinases that are active within cells under specific conditions, the binding group may be selected to have a broad, general affinity for many different types and/or classes of kinases. In other applications, where the KABP is used as a selective, chemical handle to facilitate purification of a particular kinase or family of kinases, the binding group may be selected to have very narrow, specific affinity for the target kinase or kinase family. In research directed to discover off-target interactions of a candidate therapeutic drug with kinases, the binding group may be the candidate therapeutic drug, or a derivative of the candidate therapeutic drug.

The reactive group is generally covalently bound to the binding group, and must be carefully selected according to a number of criteria. First, the reactive group needs to include one or more sufficiently reactive chemical moieties to react with kinase amino-acid side chains or, less commonly, reactive backbone moieties in order to covalently and irreversibly bind the KABP to the kinase, following binding by the kinase of the binding group. Suitable reactive chemical moieties include unsaturated carbon bonds proximal to electron withdrawing groups, such as acrylyl moieties, epoxides, azides, sulphonates, fluorophosphates, vinyl sulfones, azirines, and other reactive groups that can serve as good targets for nucleophilic addiction by amino-acid-side-chain nucleophiles. It is also possible that, in particular cases, the reactive group may tightly, but non-covalently bind at a site proximal to the binding-group binding site in order to produce, together with binding of the binding group, and possibly by inducing a conformational change in the kinase, a sufficiently low disassociation constant for the binding-group/reactive-group/kinase complex to effectively irreversibly bind to the kinase. On the other hand, the reactive group should not be so reactive that it facilitates non-specific binding of the KABP to the target kinase or to the myriad other biomolecules potentially encountered by the KABP during passive diffusion or active transport into a cell, and diffusion or active-transport-based migration of the KABP to the local environment of the target kinase within the cell. Otherwise, an overly reactive reactive group may lead to general, non-specific labeling by the KABP of kinases, whether or not active, and to various types of biopolymers and even small molecules unrelated to kinases. Such non-targeted reactions both decrease the effective concentration of the KABP within the local environment of the kinase, interfering with kinase labeling and detection, and also may produce false positive results due to the KABP binding to biopolymers unrelated to kinases or to inactive kinases that would, under normal circumstances, not bind the substrate-analog binding group of the KABP. The reactive group must also be positioned with respect to the binding group to allow the chemically reactive moiety or moieties of the reactive group to be appropriately positioned with respect to kinase functional groups following binding of the binding group within the binding domain. Thus, the covalent linkage between the reactive group and binding group needs to be of a sufficient size and conformational rigidity or flexibility to correctly position the reactive group with respect to reactive kinase moieties. The reactive group must also be linked in a way that the reactive group does not significantly alter or decrease the affinity of the kinase for the binding group. For example, conformations in which the reactive group may sterically hinder binding of the binding group, or may bind through non-covalent interactions with kinase side chains prior to positioning of the binding group within the binding domain, may greatly decrease the labeling efficiency and specificity of the KABP.

The linker group 606 is generally chosen to be relatively chemically neutral, with a length generally within an optimal spacer length range of between ten and 150 angstroms, with solubility, hydrophobicity, and conformational rigidity and flexibility that allows the linker to have reasonable permeability in cell membranes while maintaining a desired spacing between the tag group 608 and the binding and reactive group 602 and 604 in the chemical environments in which the KABP encounters target kinases. Suitable linker groups include various bisamine polyether groups, such as polyethylene glycol.

The tag group 608 may also, like the binding group, be selected based on different criteria for different applications. For example, the tag group may serve as a chemical handle to allow for binding of the tag group by an affinity-chromatography matrix or other biopolymer or compound in order to allow for subsequent purification and identification of kinase-KABP complexes. In other applications, where instrumental detection of kinase-KABP complexes is needed following various preparative steps, the tag group may be any of a variety of fluorescent, chemoluminescent, phosphorescent, or other signal-producing groups, such as biotin, a biotin derivative, synthetic fluorescent dyes, including BODIPY dyes, such as 5,7-dimethylborondipyrromethenedifluoride, or mass tags with comparatively heavy atoms that provide readily detected signatures in mass spectrograms, substrates for chemoluminescent reactions, or radioisotope labels that produce detectable α, β, or γ emissions.

Overall, a KABP 600 needs to exhibit low reactivity and affinity for non-target biomolecules encountered by the KABP, a relatively low molecular weight, to facilitate passive diffusion or active transport of the KABP into a cell, and solubility and permeability characteristics that allow the KABP to reach the local environment of target kinases in sufficient concentration to bind to, and label, the target kinases. Other desirable characteristics for KABPs include modular chemical synthesis from commercially available reagents, economical synthesis, low cellular toxicity, and, in specific applications, the ability to readily wash KAPB, not bound to kinase(s), from cellular material.

While labeling of kinases within cells is one intended application for the KABPs that represent embodiments of the present invention, it is not the only application. KABPs may also be used for labeling, identifying, and purifying kinases from extracellular environments, such as blood plasma or other biological fluids, or may possibly be used in various instrumental and biochemical processes and apparatuses for analysis of cell extracts and extracellular fluids. As briefly mentioned above, the reactive group may target chemical moieties within or near a substrate binding site or allosteric regulator binding site, and may covalently bind to amino-acid-side chains or backbone moieties, regardless of whether the backbone moieties or amino-acid-side chains are involved in the phosphoryl-group transfer reaction or substrate and regulator binding, provided that the reactive group does not significantly decrease the binding affinity of the binding group for the target binding domain of the kinase.

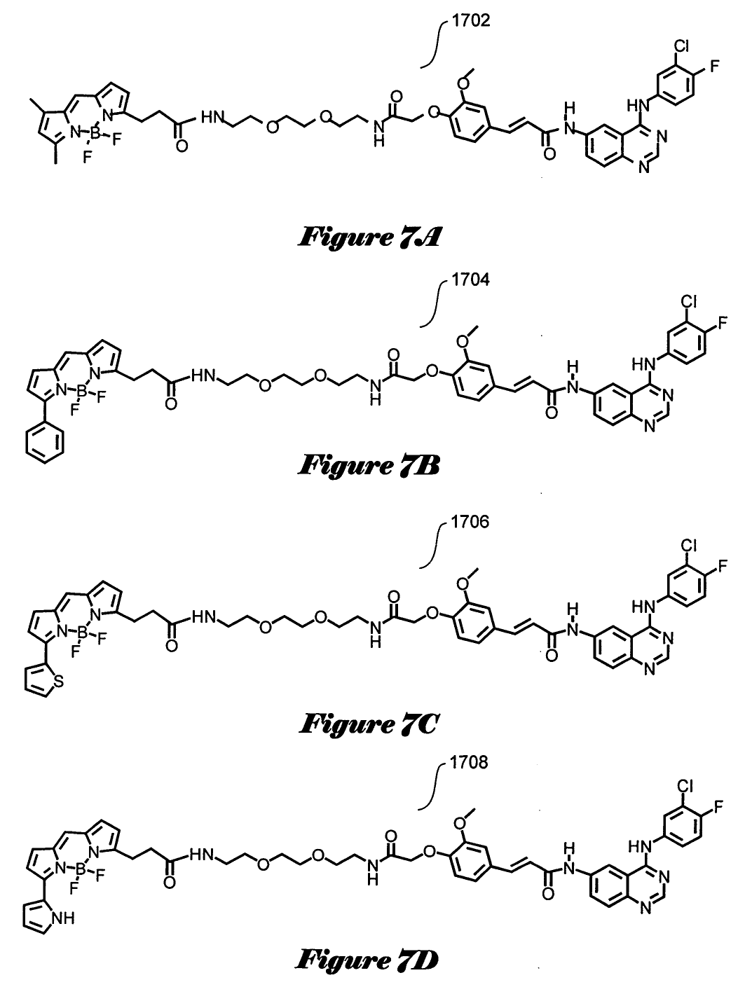

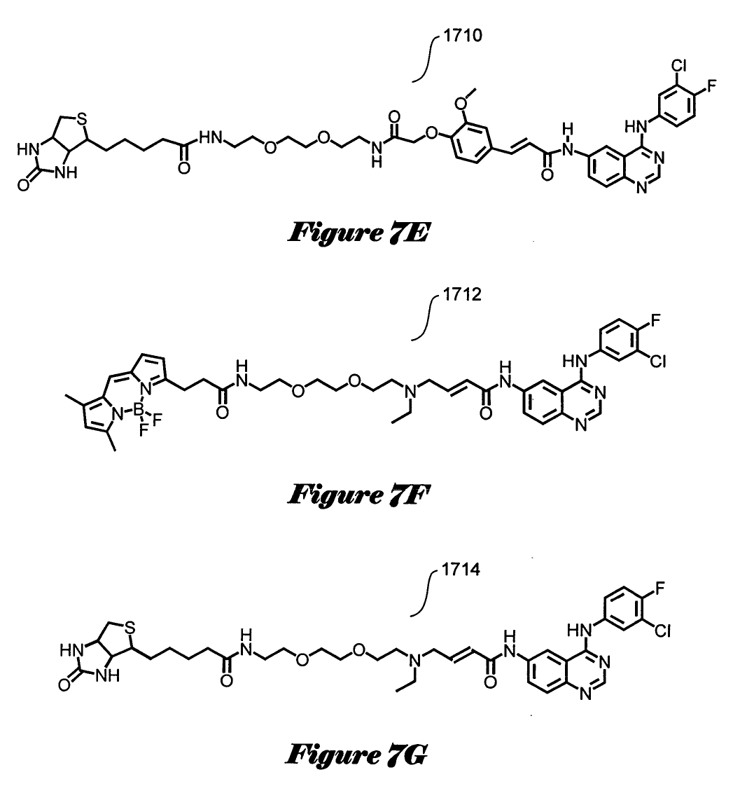

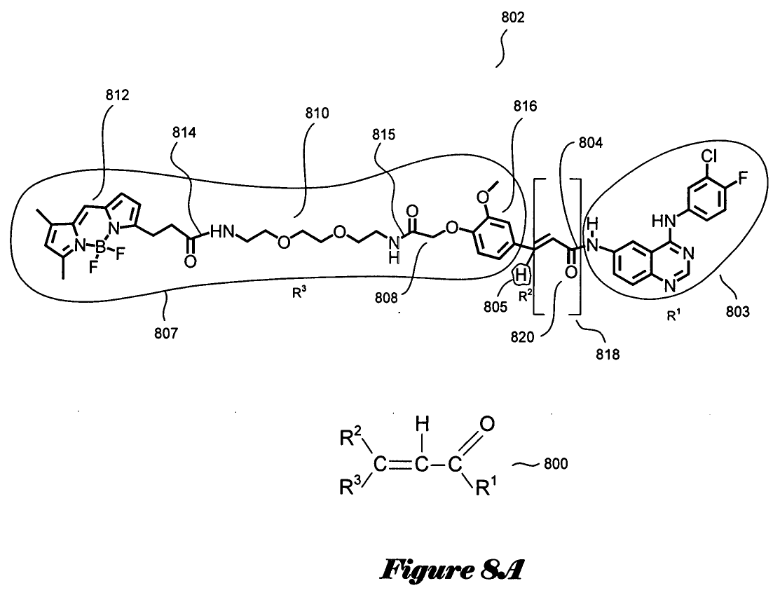

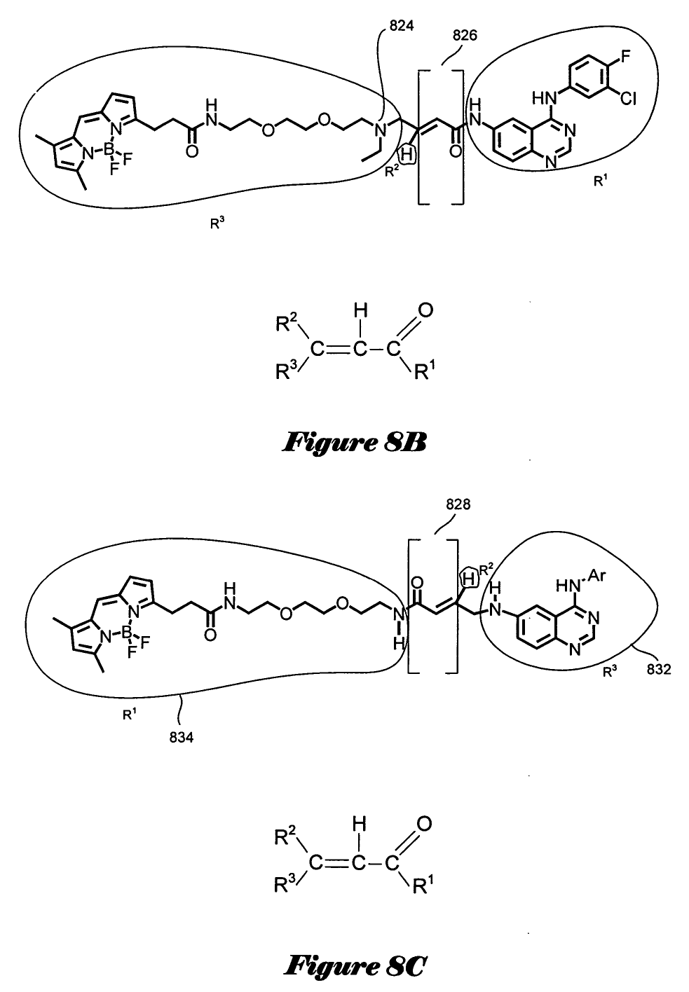

FIGS. 7A-F show the chemical structures of seven kinase-directed, activity-based probes that represent exemplary embodiments of the present invention. FIGS. 8A-C show three generalized chemical formulas for three classes of kinase-directed, activity-based probes, two of which include the specific probe molecules shown in FIGS. 7A-F, which represent embodiments of the present invention. The generalized formula shown in FIG. 8A 800 represents a class of KABPs that include the specific KABP embodiments shown in FIGS. 7A-D. The generalized formula 800 is a substituted acrylyl group, shown in brackets in FIGS. 8A-C, with substituent groups R¹, R², and R³ mapped, in FIG. 8A, to exemplary portions of the specific, example KABP 802 shown in FIG. 7A. The R¹ group 803 is the binding group (602 in FIG. 6) which, in the exemplary KABP 802, includes a nitrogen 804 linked to the carbonyl 806 of the acrylyl moiety through an amide bond. An amide linkage between binding group R¹ and the acrylyl carbonyl is but one example of different types of possible linkages, which may include ester linkages, acyl halogen linkages, and other types of linkages. In the exemplary KABPs shown in FIGS. 7A-F, the binding group is a substituted anilinoquinazoline. A variety of different substituted anilinoquinazolines are discussed below. A variety of other types of small-molecule kinase inhibitors that bind to substrate binding sites may also be used for the binding group, and examples of other types of small-molecule kinase inhibitors that can serve as binding groups of KABPs are also discussed below. In evaluating potential off-target kinase interactions of candidate therapeutic drug compounds, the therapeutic drug compound, or a derivative of the therapeutic drug compound suitable for linking to the acrylyl carbonyl may also be used as a binding group. Additional binding groups may be compounds closely related to the natural substrates for the target kinase, including nucleotides and nucleotide derivatives, saccharides and polysaccharides, peptides and polypeptides, and small-organic-molecule metabolites. However, in many applications, small-molecule aromatics, polycyclic, and heterocyclic compounds provide more favorable membrane permeability for the KABP in which they are included, in turn providing KABPs more suitable for labeling kinases within intact cells.

The R² group 805 is, in the exemplary embodiment 802, a hydrogen atom. In alternative embodiments, the R² group may be any of numerous substituents, including halogen atoms, alkyl groups, a substituted alkyl group, and more complex, carbon based groups that include double and triple bonds.

The R³ group 807 includes a portion of the reactive group (604 in FIG. 6), the entire linker group (606 in FIG. 6), and the entire tag group (608 in FIG. 6) of the exemplary KABP 802. The portion of the reactive group in the exemplary R³ group shown in FIG. 8A is a methoxy, glycolyl bisubstituted phenyl moiety. The glycolyl carbonyl 808 is linked through an amide bond to a {2-[2-(2-amino-ethoxy)-ethoxy]-ethyl}-carbamyl moiety 810, in turn linked to a fluorescent tag molecule 812. As discussed above, the linker group may be a polyethylene-glycol-based polyether, or another polymer, such as substituted and unsubstituted polyethylenyl, polypropyleneyl, and polyaminyl polymers having lengths suitable for spacing the tag group 812 from the active and binding groups and/or conformational. rigidity to prevent looping of the KABP resulting in association of the binding/reactive groups and the tag group. In most applications, the KAPBP needs to be water soluble, so linker groups preferably contain oxygen, nitrogen, or other atoms that can for hydrogen bonds with solvent molecules, or that are ionizable or sufficiently polar to provide reasonable water solubility. As discussed above, any of a variety of commercially available or novel tag groups can be incorporated into KABP embodiments, depending on the intended application for the KABP. Tag groups generally either emit an instrumentally detectable signal, such as fluorescent, phosphorescent, or chemoluminescent emission of photons or radioactive alpha, beta, or gamma emission, include elements that are easily detectable, by mass, using mass-spectroscopy methods, or serve as a chemical handle that can be recognized and bound to specific compounds or macromolecules in subsequent procedures to facilitate isolation and purification of kinase-KABP pairs or to subsequently generate an instrumentally detectable signal as a result of binding of the tag molecule to a compound or macromolecule that emits a detectable signal when associated with the tag.

The reactive group of the exemplary KABP 802 is the acrylyl moiety, which is readily attacked by nucleophiles, such as cysteinyl sulfhydryls, at the carbon-carbon double bond 814. Nucleophilic attack is facilitated by a conjugated electron-withdrawing carbonyl 806 and the methoxy, glycolyl-hydroxy bisubstituted phenyl 816. Many additional reactive moieties may be employed within various different reactive groups for covalent binding with kinase functional groups, including epoxides, azerines, azides, sulphonates, fluorophosphates, vinyl sulfones, isonitriles, and other relatively reactive groups. As discussed above, the reactive group needs to readily react with a kinase moiety once the binding group is bound by the kinase, but also needs to not be so reactive that reactions readily occur with other biomolecules encountered by the KABP prior to KABP interaction with the kinase, or non-specific binding of the KABP to target kinases occurs prior to binding of the binding group by the target kinase. Also, both reactive and linker linkages to the binding group need to be designed to prevent a decrease in kinase affinity for the binding group and destabilization of the kinase-binding group complex.

FIG. 8B shows a general formula for a second family of exemplary kinase-directed, activity-based probes. The general formula 820 in FIG. 8B is identical to the general formula shown in FIG. 8A, but, as indicated by the mapping between the R groups of the general formula and an exemplary KABP 822, the R³ group 823 is a {2-[4-(3-ethylamino-ethoxy)-ethoxy]-ethyl}-carbamyl moiety linked to a tag group. The class of KABPs represented by the generalized formula in FIG. 8B includes the exemplary KABPs shown in FIGS. 7D and 7F.

FIG. 8C shows a generalized formula for a third class of kinase-directed, activity-based probes. In this class of probes, the orientation of the acrylyl moiety 830 is reversed from the orientation of the acrylyl moieties in the first two classes of KABPs described with reference to FIGS. 8A-B, with the R³ group 832 a substituted anilinoquinazoline and the R¹ group 834 is a {2-[4-(3-ethylamino-ethoxy)-ethoxy]-ethyl}-carbamyl moiety linked to a tag group. Alternatively, all three classes of KABPs discussed with reference to FIGS. 8A-C can be described with the single general formula 828, where it is understood that the R¹ and R³ groups may be interchanged.

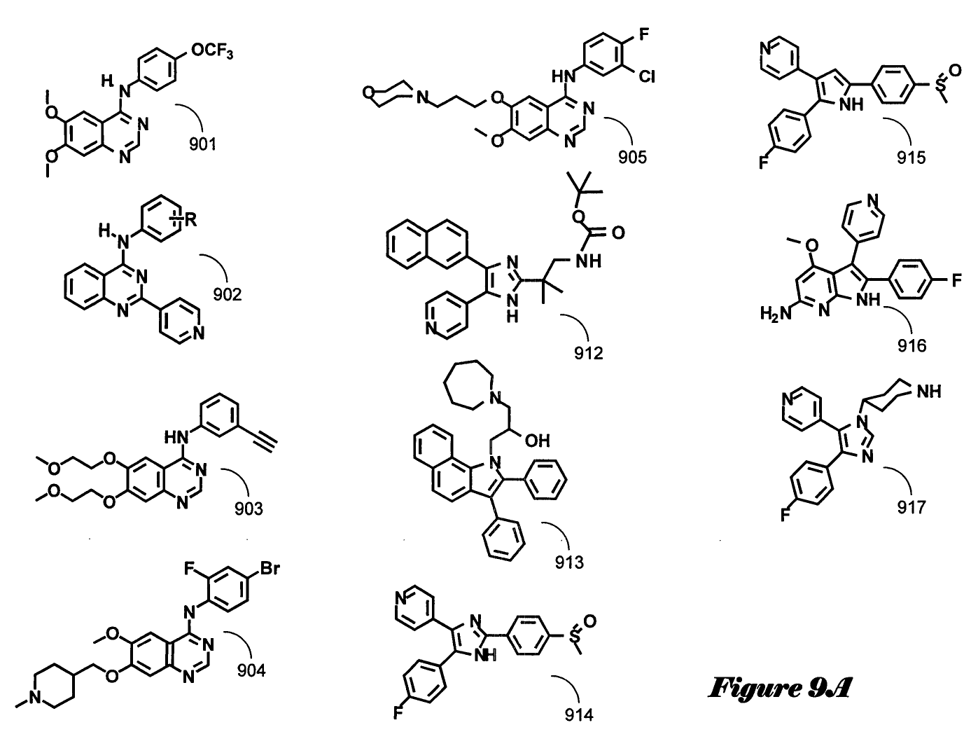

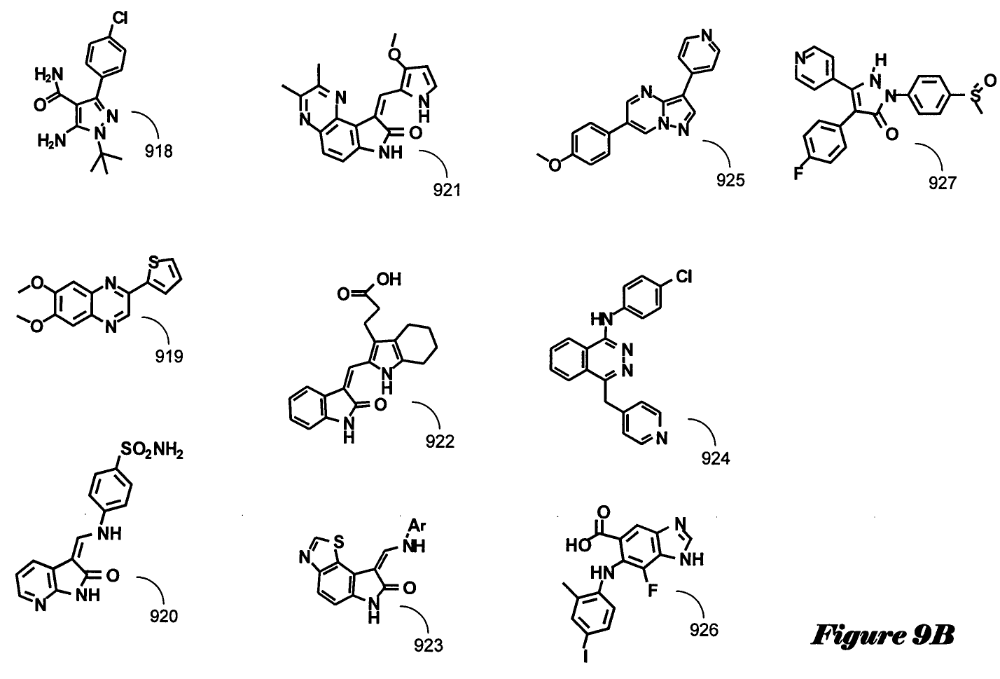

FIG. 9 shows a number of different small-molecule inhibitors, including known kinase inhibitors, that may be used, either in the forms shown in FIG. 9, or in derivative forms, as binding groups for alternative KABP embodiments of the present invention. As can be seen by examination of the chemical structures 901-927 shown in FIG. 9, a wide variety of different substituted aromatic, polycyclic and heterocyclic compounds are bound with high affinity by kinases.

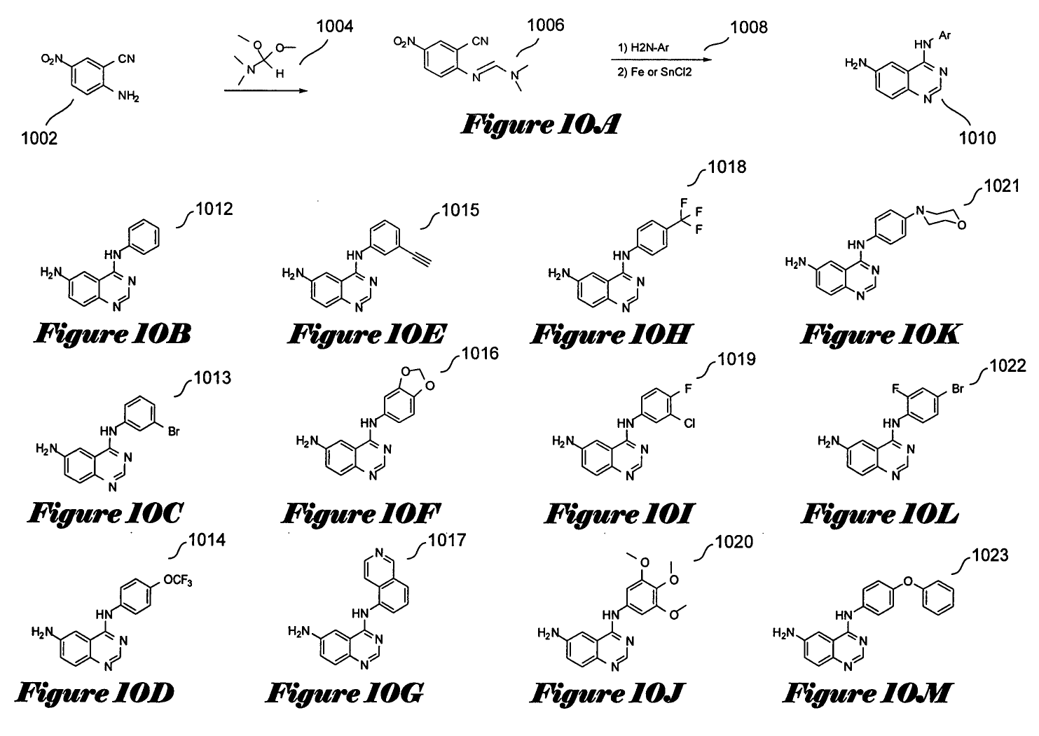

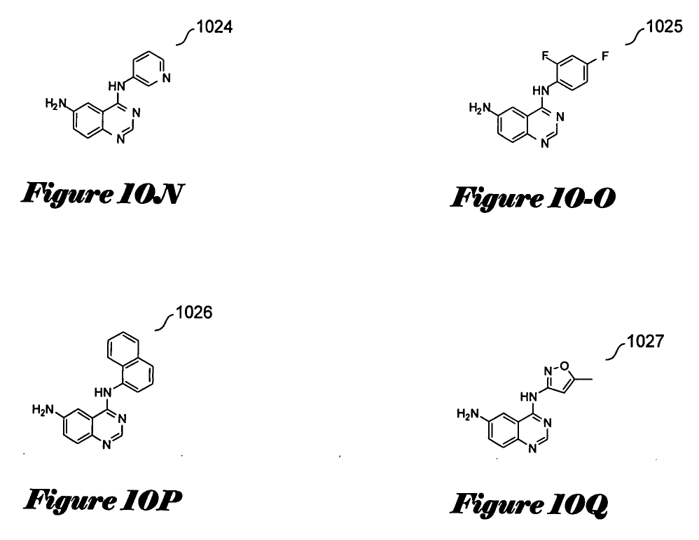

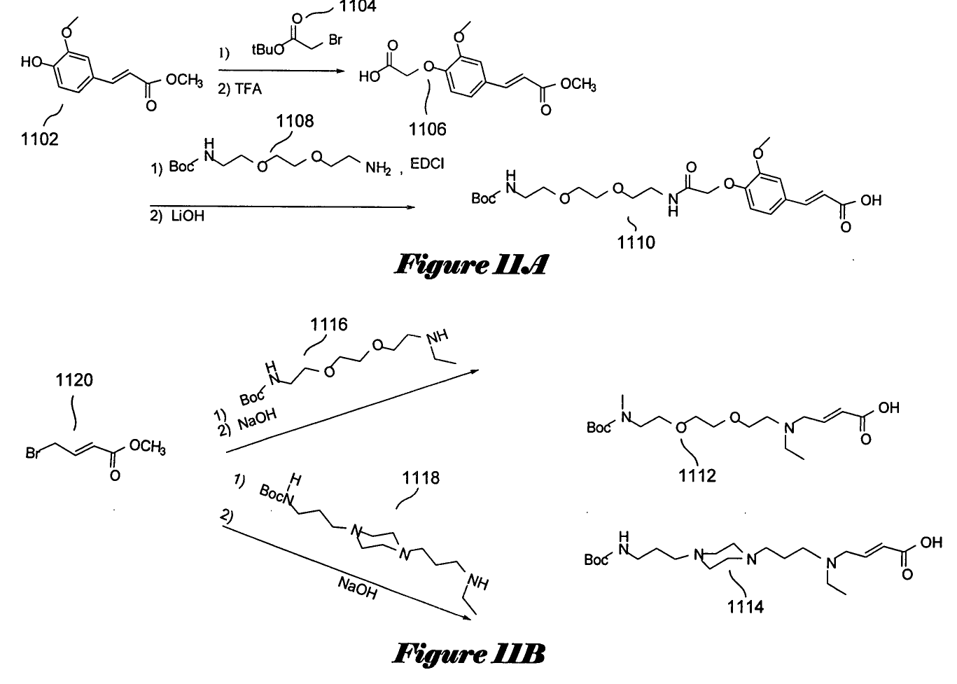

FIG. 10A illustrates a general approach for synthesis of a variety of different anilinoquinazoline moieties that can serve as binding groups within kinase-directed, activity-based probes that represent embodiments of the present invention. In the synthetic steps shown in FIG. 10A, a 2-amino-5-nitrobenzonitrile 1002 is refluxed in N,N-dimethylformamide dimethyl acetal 104 to produce N′-(4-nitro-2-cyano-phenyl)-N,N-dimethyl-formamidine 1006 which is then warmed in acetic acid with one of various different aliphatic or aromatic substituted amines 1008 to produce N⁴-substituted 6-nitro quinazolines, in turn reduced, using SnCl₂ or FeCl₃, to produce N⁴-substituted-quinazoline-4,6-diamines 1010. FIGS. 10B-10N show a number of different N⁴-substituted quinazoline-4,6-diamines produced by the synthetic steps shown in FIG. 10A.

FIGS. 11A-B illustrate several alternative synthetic methods for synthesizing reactive-groups/linker-group moieties included in kinase-directed, activity-based probes that represent embodiments of the present invention. FIG. 11 shows a reactive-group/linker-group synthesis. Methyl 4-hydroxy-3-methoxycinnamate 1102 is alkylated with tert-butyl bromoacetate 1104 and the resulting tert-butyl ester is cleaved with trifluoroacetic acid to produce the intermediate compound 1106. The intermediate compound is then treated with 1-(3-diethylaminopropyl)-3-ethylcarbodiimide (“EDCI”) and N-hydroxy succinimide, and {2-[2-(2-amino-ethoxy)-ethoxy]-ethyl}-carbamic acid tert-butyl ester 1108 is added to the resulting intermediate activated ester, and the product saponified, to produce a first reactive-group/linker-group moiety 1110. In FIG. 11B, two alternative reactive-group/linker-group moieties 1112 and 1114 are prepared by alkylation of {2-[2-(2-ethylamino-ethoxy)-ethoxy]-ethyl}-carbamic acid tert-butyl ester 1116 or {3-[4-(3-ethylamino-propyl)-piperazin-1-yl]-propyl}-carbamic acid tert-butyl ester 1118 with 4-bromo-butenoic acid methyl ester 1120.

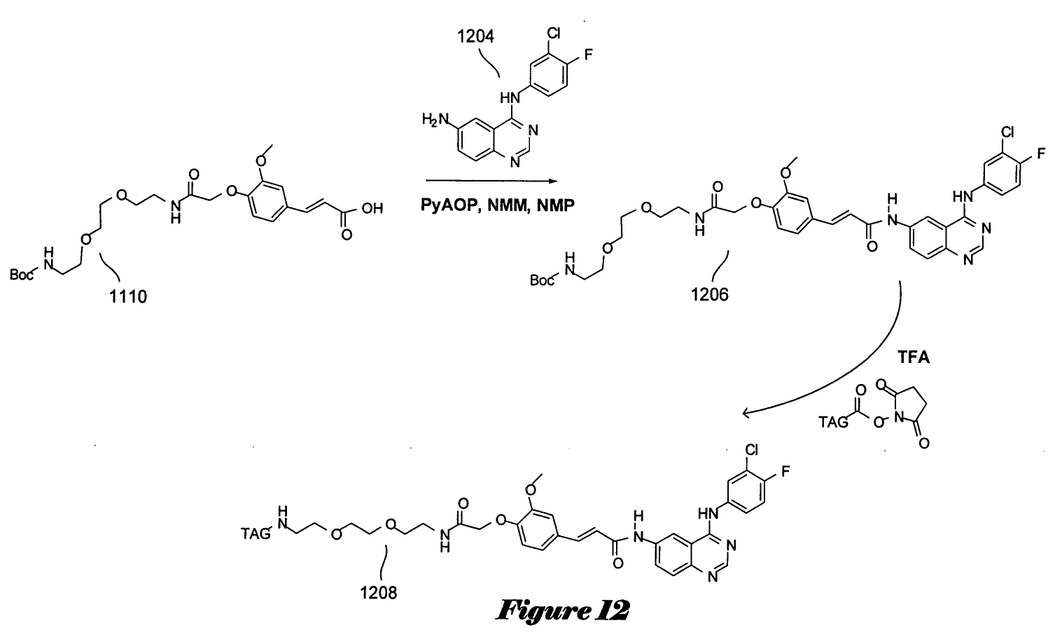

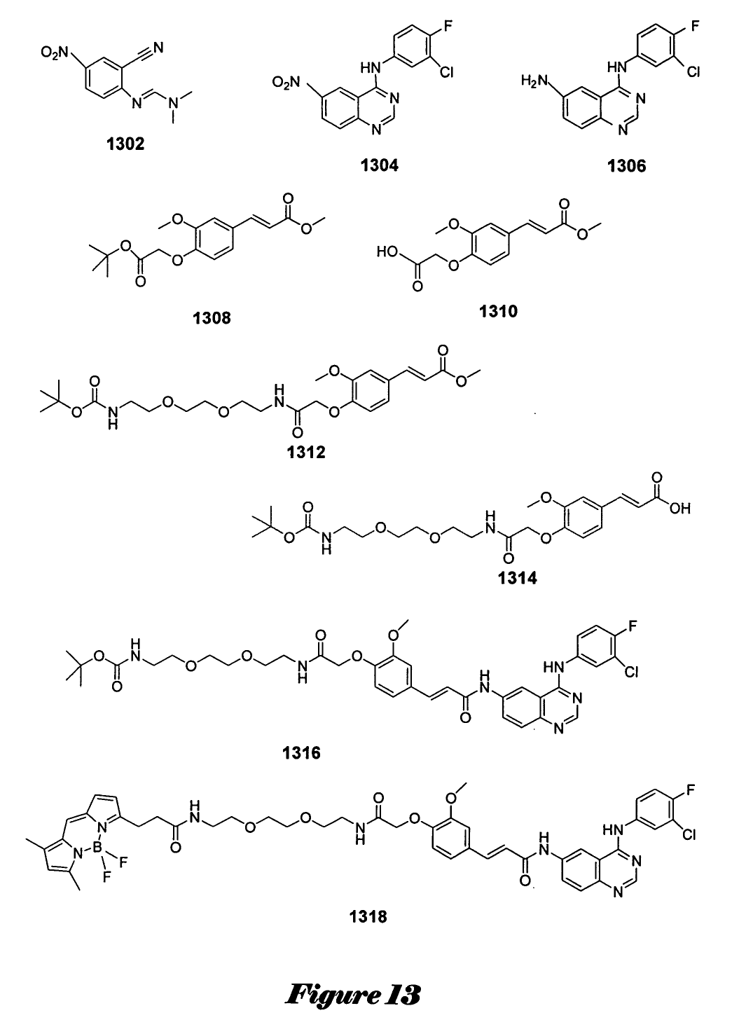

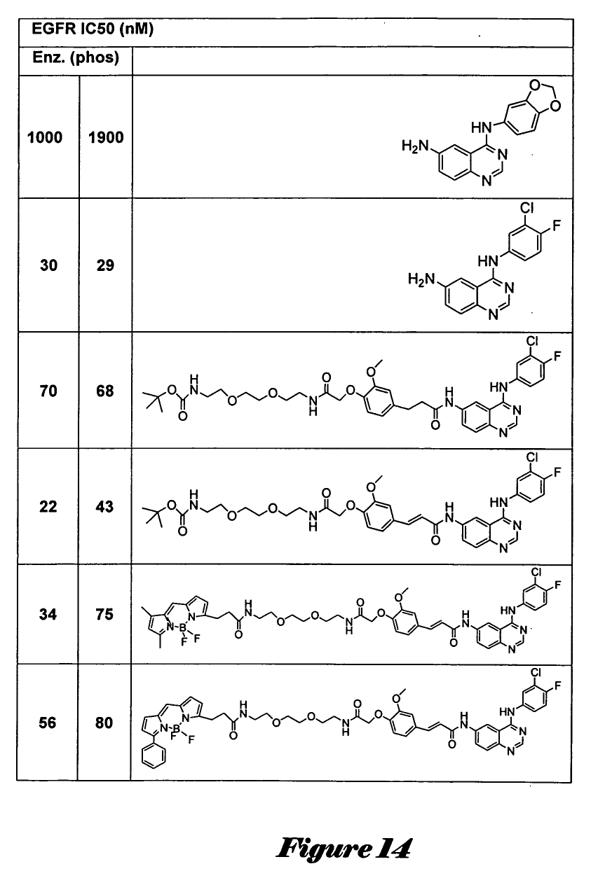

FIG. 12 shows final synthetic steps used to assemble an exemplary kinase-directed, activity-based probe that represents one embodiment of the present invention. A reactive-group/linker-group intermediate 1202 prepared by the synthetic steps shown in FIG. 11A is esterified with 7-azabenzotriazol-1-yl-oxy-tris-pyrrolidino-phosphonium (“PyAOP”) in 1-methyl-2-pyrrolidinone (“NMP”) and N-methylmorpholine (“NMM”) to produce an intermediate ester which is then reacted with an anilinoquinazoline 1204 binding group to produce a linker-group/reactive-group/binding-group intermediate 1206. The intermediate 1206 is then treated with trifluoroacetic acid to produce a primary amine by removing the tert-butyl ester group, and the primary amine is then reacted with a tag succinate ester, such as a BODIPY-succinate ester or a d-biotinyl succinate ester, to produce a final KABP 1208. Clearly, a huge variety of KABPs can be synthesized by these general procedures using different binding groups, linking groups, tag groups, and reactive groups.

Although the present invention has been described in terms of particular embodiments, it is not intended that the invention be limited to this embodiment. Modifications within the spirit of the invention will be apparent to those skilled in the art. For example, as discussed above, a very large number of different KABPs can be synthesized for different applications by combinatorial synthesis of KABPs using a variety of different tag-group, linker-group, reactive-group, and binding-group modules. Although, in above-disclosed embodiments, the linker group is covalently bound to the reactive group and the tag group, the linker group may, in alternative embodiments, be covalently bound to one or both of the reactive and binding groups, on a first end, and the tag group, on a second end, to space the binding and reactive groups apart from the tag group. The detailed synthetic steps needed for linking the various different modular components together may vary, depending on the exact chemistries of the modular components. KABPs can be used for a variety of different purposes and in a variety of different applications. As discussed above, KABPs can be used to label active kinases within cells, for subsequent identification, isolation, and purification, and can be used in a variety of preparative and analytical procedures in which soluble kinases are identified in solutions, isolated and purified from solutions, or otherwise investigated or studied. KABPs may be used, with candidate therapeutic drug binding groups, or derivatized candidate therapeutic drug binding groups, in order to investigate interaction of the candidate drug with kinases within intact cells, cell-extract solutions, or other kinase-containing systems. KABPs with binding groups having broad affinity for many different kinases and kinase families can be used to search for, and identify, new, as yet undiscovered kinases, or to determine when, in different points of the cell cycle, or in different cellular environments, various kinases are activated. For example, the kinase-based mechanisms by which small-molecule stimulants exercise influence on cellular mechanisms may be investigated using KABP labels having binding groups with different specificities for different kinases, and introduced at different points in time following exposure of cells to the small-molecule stimulant. KABPs may also be used as components in various analytical and diagnostic processes and instrument-based methods for ascertaining kinase activities in various sample solutions.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A kinase-directed, activity-based probe with a molecular weight of between 500 and 2500 that binds to one or more target kinases, the kinase-directed, activity-based probe comprising:
a binding moiety that binds to one of
a substrate binding site of the one or more target kinases, and
an allosteric-regulator binding site of the one or more target kinases;
a reactive moiety covalently linked to the binding moiety that reacts with a kinase;
a tag moiety that provides one of
an instrumentally detectable signal, and
a chemical handle that is recognized and bound by a chemical compound, macromolecule, or substrate material; and
a linker moiety that covalently links the tag moiety to one or both of the binding and reactive moieties.

2. The kinase-directed, activity-based probe of claim 1 wherein the binding moiety is a substituted anilinoquinazoline.

3. The kinase-directed, activity-based probe of claim 2 wherein the anilinoquinazoline is selected from the anilinoquinazolines shown in FIGS. 10B-N.

4. The kinase-directed, activity-based probe of claim 1 wherein the binding moiety is a small-organic-molecule inhibitor of the one or more target kinases.

5. The kinase-directed, activity-based probe of claim 4 wherein the small-organic-molecule competitive inhibitor is one of the kinase competitive inhibitors shown in FIG. 9.

5. The kinase-directed, activity-based probe of claim 5 wherein the small-organic-molecule competitive inhibitor is a derivative of one of the kinase competitive inhibitors shown in FIG. 9.

7. The kinase-directed, activity-based probe of claim 1 wherein the binding moiety is a small-organic-molecule candidate therapeutic drug or small-organic-molecule-candidate-therapeutic-drug derivative that may bind to the one or more target kinases.

8. The kinase-directed, activity-based probe of claim 1 wherein the reactive moiety includes a reactive bond or functional group selected from among:
an unsaturated carbon-carbon bond conjugated with an electron-withdrawing atom or group;
an epoxide,
an azerine,
an azide,
a sulphonate,
a fluorophosphate,
a vinyl sulfone, and
an isonitrile.

9. The kinase-directed, activity-based probe of claim 1 wherein the linker moiety is a polyethylenyl, polypropyl, polyaminyl, or other polyether, with terminal amine nitrogens that link the linker moiety through amide bonds to the tag moiety and one or both of the linker and reactive moieties.

10. The kinase-directed, activity-based probe of claim 1 wherein the tag moiety is a signal producing group that produces an instrumentally detectable signal selected from among:
fluorescent emission;
phosphorescent emission;
chemiluminescent emission;
α emission;
β emission; and
γ emission.

11. The kinase-directed, activity-based probe of claim 10 wherein the tag moiety is selected from among:
a BODIPY fluorescent dye; and/or
biotin.

12. The kinase-directed, activity-based probe of claim 1 wherein the tag moiety includes one or more atoms with masses easily identified by mass spectroscopy.

13. The kinase-directed, activity-based probe of claim 1 wherein the tag moiety is a chemical handle selected from among:
a moiety that is bound by an affinity-chromatography matrix;
a substrate for a chemiluminescence-producing reaction;
a moiety that binds a small-molecule compound or macromolecule complexing agent to form a kinase-directed-activity-based-probe/kinase/complexing-agent trinary complex used to isolate or identify the one or more target kinases.

14. A kinase-directed, activity-based probe comprising a substituted acrylyl moiety having the structure R³—,R²—C═C—CO,—R¹ wherein:
R³ is selected from among
a methoxy, glycolyl-hydroxy bisubstituted phenyl linked through an amide bond to a 2-[2-(2-amino-ethoxy)-ethoxy]-ethyl amine, in turn linked through an amide bond to a fluorophore tag group, and
an N-alkylated 2-[2-(2-amino-ethoxy)-ethoxy]-ethyl amine linked through an amide bond to a fluorophore tag group;
R² is selected from among
a hydrogen atom,
a halogen atom,
an alkyl group, and
a substituted alkyl group; and
R¹ is selected from among
a substituted anilinoquinazoline,
a competitive kinase inhibitor, and
a candidate therapeutic drug.

15. The kinase-directed, activity-based probe of claim 14 wherein R¹ is selected from among:
an anilinoquinazoline shown in one of FIGS. 10B-10N; and
a kinase competitive inhibitor, or derivative thereof, shown in FIG. 9.

16. A kinase-directed, activity-based probe comprising a substituted acrylyl moiety having the structure R³—,R²—C═C—CO,—R¹ wherein:
R¹ is selected from among
a methoxy, glycolyl-hydroxy bisubstituted phenyl inked through an amide bond to a 2-[2-(2-amino-ethoxy)-ethoxy]-ethyl amine, in turn linked through an amide bond to a fluorophore tag group, and
an N-alkylated 2-[2-(2-amino-ethoxy)-ethoxy]-ethyl amine linked through an amide bond to a fluorophore tag group;
R² is selected from among
a hydrogen atom,
a halogen atom,
an alkyl group, and
a substituted alkyl group; and
R³ is selected from among
a substituted anilinoquinazoline,
a competitive kinase inhibitor, and
a candidate therapeutic drug.

17. The kinase-directed, activity-based probe of claim 16 wherein R³ is selected from among:
an anilinoquinazoline shown in one of FIGS. 10B-10N; and
a kinase competitive inhibitor, or derivative thereof, shown in FIG. 9.

18. A kinase-directed, activity-based probe that irreversibly binds one or more target kinases selected from among the kinase-directed, activity-based probes shown in FIGS. 7A-F.

19. A method for labeling one or more kinases within an intact cell that actively bind a substrate analog, the method comprising:
providing a kinase-directed, activity-based probe directed to the one or more kinases;
exposing the cell to the kinase-directed, activity-based probe; and
processing the cell.

20. The method of claim 19 wherein the kinase-directed, activity-based probe, directed to the one or more kinases, comprises:
a binding moiety that binds to one of
a substrate binding site of the one or more target kinases, and
an allosteric-regulator binding site of the one or more target kinases;
a reactive moiety covalently linked to the binding moiety that reacts with a kinase
a tag moiety that provides one of p2 an instrumentally detectable signal, and
a chemical handle that is recognized and bound by a chemical compound, macromolecule, or substrate material; and
a linker moiety that covalently links the tag moiety to one or both of the binding and reactive moieties.

21. The method of claim 20 wherein the tag moiety is one of:
a substituted anilinoquinazoline;
an anilinoquinazoline selected from the anilinoquinazolines shown in FIGS. 10B-N;
a small-organic-molecule inhibitor of the one or more target kinases;
a small-organic-molecule competitive inhibitor selected from the competitive inhibitors shown in FIG. 9;
a small-organic-molecule competitive-inhibitor derivative of one of the kinase competitive inhibitors shown in FIG. 9; and
a small-organic-molecule candidate therapeutic drug or small-organic-molecule-candidate-therapeutic-drug derivative that may bind to the one or more target kinases.

22. The method of claim 19 wherein exposing the cell to the kinase-directed, activity-based probe further comprises:
introducing the kinase-directed, activity-based probe into a medium surrounding the cell at sufficient concentration to allow for one of
the kinase-directed, activity-based probe to be actively transported into the cell, and
the kinase-directed, activity-based probe to diffuse into the cell;
waiting for a sufficient period of time to allow the kinase-directed, activity-based probe to irreversibly bind to the one or more kinases; and
removing remaining kinase-directed, activity-based probe from the medium surrounding the cell.

23. The method of claim 19 wherein processing the cell further comprises:
lysing the cell and extracting cellular contents into a solution;
processing the solution to at least partially purify the one or more kinases; and
instrumentally detecting a signal from the at least partially purified one or more kinases.

24. The method of claim 23 wherein the detected signal is one of:
fluorescent emission;
phosphorescent emission;
chemiluminescent emission;
α emission;
β emission; and
γ emission.
